(19) United States
(12) Patent Application Publication
(10) Pub. No.: US 2006/0173285 A1
(43) Pub. Date: Aug. 3, 2006

(54) Methods and systems for reducing RF-induced heating in magnetic resonance imaging

(75) Inventors: Richard Philip Mallozzi, Ballston Lake, NY (US); Charles Lucian Dumoulin, Ballston Lake, NY (US); Patrick Gross, Muenchen (DE)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 11/017,466

(22) Filed: Dec. 20, 2004

(51) Int. Cl. A61B 5/05

(52) U.S. Cl. 600/422

(57) ABSTRACT

A conducting wire assembly (80) is provided for use with a magnetic resonance imaging (MRI) system (10). The conducting wire assembly (80) includes at least one impedance component (82) coupled externally to a conducting wire (34). The impedance component (82) is configured to dynamically vary an impedance of the conducting wire to disrupt resonant conditions of the conducting wire and to avoid current and/or voltage built-up on the wire and the associated heating of surrounding tissue.

### BACKGROUND

The invention relates generally to interventional imaging, and in particular to systems and methods of reducing RF-induced heating in magnetic resonance imaging using invasive devices.

In magnetic resonance (MR) imaging systems using invasive devices, large electrical currents and/or voltages may be created on long wire-like conducting structures of the invasive device, for example a catheter, placed in an MR scanner. A standing radio frequency (RF) wave is generated along the length of the conducting wire due to the resonant conditions that occur by the interaction of the conducting wire with RF fields of the imaging system. The resonant conditions lead to a built-up of large currents and/or voltages and potentially dangerous heating effects, both for the device and for the surrounding tissue.

Typically choking circuits have been placed along the length of the wire to disrupt these large currents and/or voltages. Alternately, an electrically conductive shield has been proposed for the length of the conducting wire, wherein the electrical shield has an electric resistance, which is substantially higher than the electrical resistance of the electrical connection over the same length. The electric shield generally comprises alternately electrically high conducting portions and low conducting portions. The low conducting portions help to reduce the building-up of the standing RF wave and thus reduce heating effects. These techniques have serious fabrication issues, especially for small catheters. In another technique, resonant coils and dynamic disabling circuits have been built using a DC bias on the device to detune the coil by adding capacitors and diodes inside the coil circuitry. This adds to bulk and complexity of the device and there are safety issues due to DC bias within the device.

Therefore there is a need for a system and technique for effectively reducing the RF-induced heating due to built-up of large currents and/or voltages in long conducting structures used with the invasive devices in magnetic resonance imaging.

### BRIEF DESCRIPTION

Briefly, in accordance with one aspect of the present technique, a conducting wire assembly is provided for use with a magnetic resonance imaging (MRI) system. The conducting wire assembly includes at least one impedance component coupled externally to a conducting wire. The impedance component is configured to dynamically vary an impedance of the conducting wire to disrupt resonant conditions of the conducting wire and to avoid current and/or voltage built-up on the wire.

In accordance with another aspect, a method for reducing RF-induced heating in magnetic resonance imaging (MRI) is provided. The method includes externally coupling an impedance component to a conducting wire and dynamically varying an impedance of the conducting wire with time via the impedance component for disrupting resonant conditions in the conducting wire and for avoiding current and/or voltage built-up on the conducting wire resulting in reduced heating of the surrounding tissue.

### DRAWINGS

These and other features, aspects, and advantages of the present invention will become better understood when the following detailed description is read with reference to the accompanying drawings in which like characters represent like parts throughout the drawings, wherein:

FIG. 1 is a schematic block diagram of an exemplary magnetic resonance (MR) imaging system suitable for use with the present techniques;

FIG. 2 is a flow-chart illustrating an exemplary method for dynamically varying an impedance of a conducting wire for use with an MR system of FIG. 1;

FIG. 3 is a diagrammatical representation illustrating one aspect of the method of FIG. 2 using a time varying impedance;

FIG. 4 is a diagrammatical representation illustrating another aspect of the method of FIG. 2 using a diode for altering an electrical length of the conducting wire; and

FIG. 5 is a graphical comparison of the effect on heating of the conducting wire using standard termination and the method of FIG. 2.

### DETAILED DESCRIPTION

It is frequently desirable to place conducting structures in a magnet resonance (MR) scanner, such as ECG and pacemaker leads, catheter guide wires, and MR tracking devices for catheters. Such wire-like structures interact with the radio-frequency fields in the scanner, and under certain conditions can support large electrical currents and/or voltages and fields that can cause dangerous local heating in tissue. Aspects of the present technique provide alternative ways to keep these currents and/or voltages low by interfering with the resonant conditions that lead to the large currents and/or voltages and electric fields.

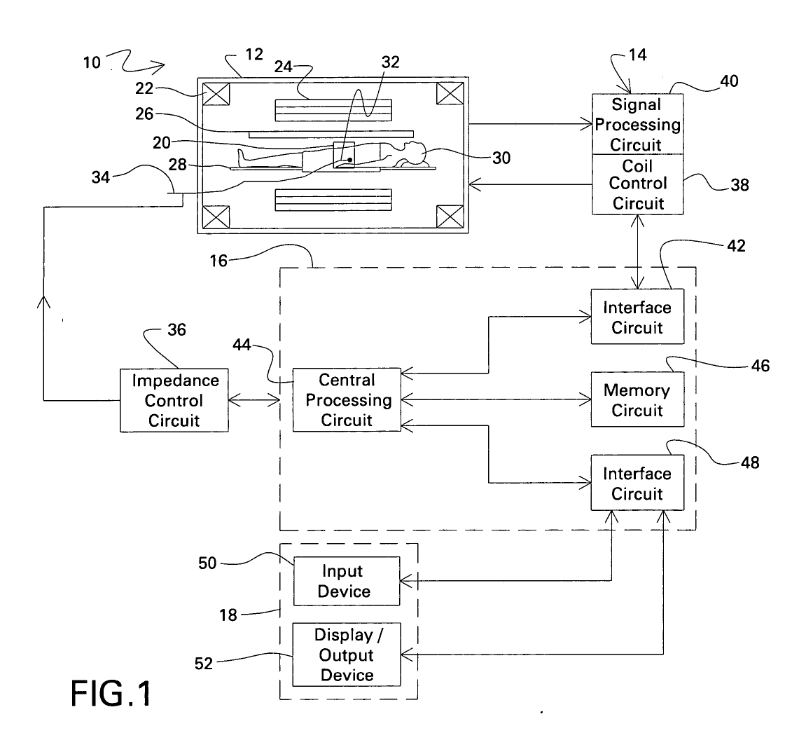

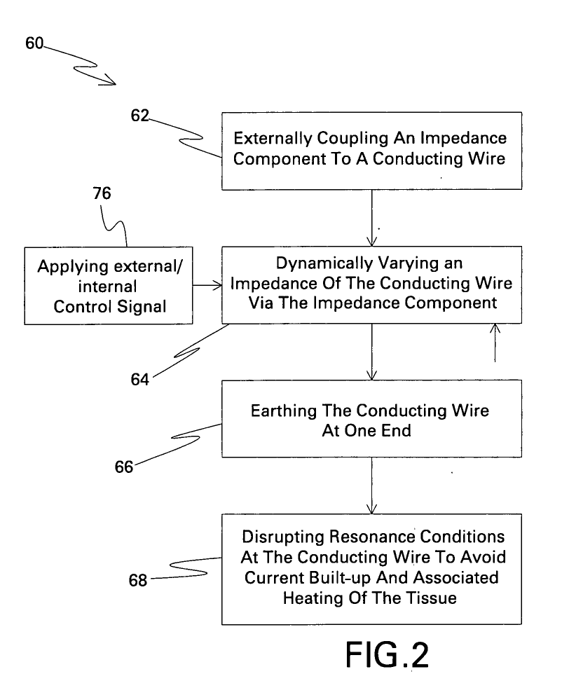

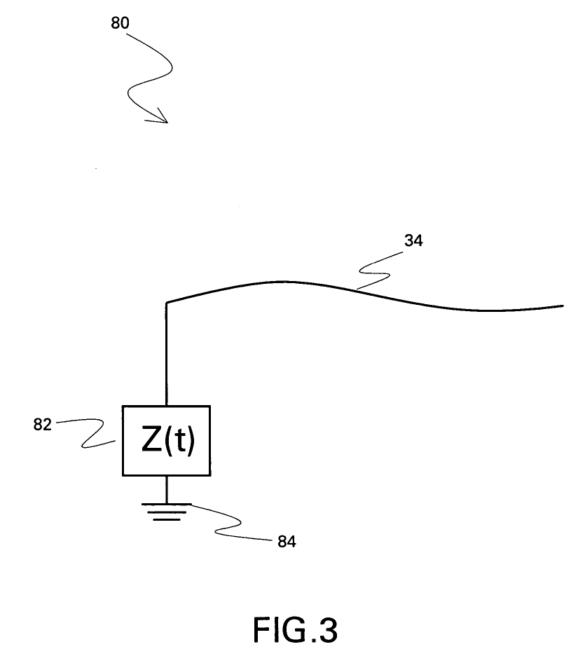

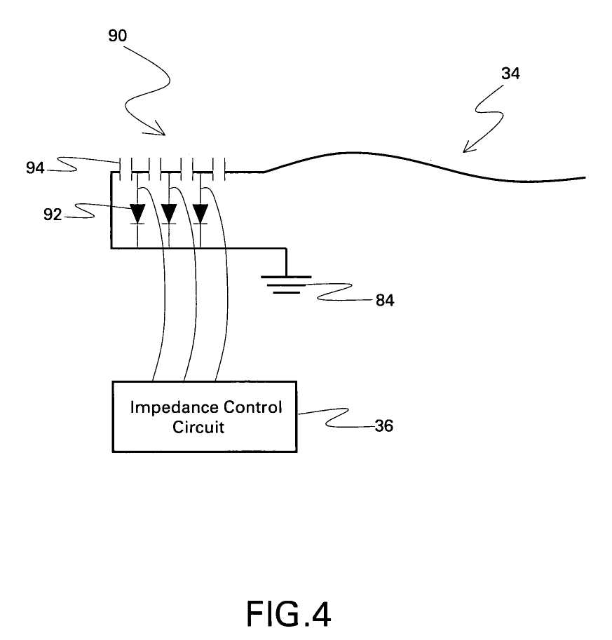

Referring now to FIG. 1, a magnetic resonance system, designated generally by the reference numeral 10, is illustrated as including a magnet assembly 12, a control and acquisition circuit 14, a system controller circuit 16, and an operator interface station 18. The magnet assembly 12, in turn, includes coil assemblies for selectively generating controlled magnetic fields used to excite gyromagnetic materials spin systems in a subject of interest. In particular, the magnet assembly 12 includes a primary coil 22, which will typically include a superconducting magnet coupled to a cryogenic refrigeration system (not shown). The primary coil 22 generates a highly uniform B0 magnetic field along a longitudinal axis of the magnet assembly. A gradient coil assembly 24 consisting of a series of gradient coils is also provided for generating controllable gradient magnetic fields having desired orientations with respect to the anatomy or region of interest 20. In particular, as will be appreciated by those skilled in the art, the gradient coil assembly produces fields in response to pulsed signals for selecting an image slice, orienting the image slice, and encoding excited gyromagnetic material spin systems within the slice to produce the desired image. In Spectroscopy systems these gradient fields may be used differently. An RF transmit coil 26 is provided for generating excitation signals that result in MR emissions from a subject 30 that are influenced by the gradient fields, and collected for analysis as described below. According to aspects of the present technique, a device 32 may be inserted or optionally disposed near the anatomy of interest 20 of the subject 30. The device 32 maybe a guidewire, a catheter, an endoscope, a laparoscope, a biopsy needle, a hand-held device, minimally invasive coil such as endorectal coil, endovaginal coil, surface coil or any other similar device. The device 32 in one example receives the MR signals generated from the anatomy of interest 20 and the signals are collected for analysis as described below. It will be understood by those skilled in the art that the device 32 may not necessarily be configured to detect MR signals. If desired, the device 32 may incorporate one or more radiofrequency (RF) coils. The device 32 also includes a conducting wire assembly 34 which is described in more detail with reference to FIG. 3 and FIG. 4. In general, large, undesirable currents and/or voltages are built up on wire-like devices that result from the wire supporting a resonant current and/or voltage oscillation that is close to the frequency of the radio frequency fields in the magnet assembly. The behavior of such resonant modes is very sensitive to boundary conditions and other electromagnetic properties of the conducting wire. The aspects of present technique as explained in reference to FIG. 2-5, include changing dynamically impedance at one end of the wire, or altering the effective electrical length of the device so that the resonant conditions are disrupted dynamically. This helps to avoid the large current and/or voltage built-up and therefore avoids heating of the tissue and of the device due to the large currents and/or voltages. An impedance control circuit 36 which may be coupled to the system controller 16 may be used to provide control signals for dynamically altering the impedance of the conducting wire 34 according to aspects of present techniques which will be described in more detail in reference to FIG. 2-5.

As used herein ‘conducting wire’ and ‘wire-like structure’ imply the same and are used interchangeably.

A table 28 is positioned within the magnet assembly 12 to support the subject 30. While a full body MRI system is illustrated in the exemplary embodiment of FIG. 1, the technique described below may be equally well applied to various alternative configurations of systems and scanners, including smaller scanners and probes used in MR applications.

In the embodiment illustrated in FIG. 1, the control and acquisition circuit 14 includes coil control circuit 38 and signal processing circuit 40. The coil control circuit 38 receives pulse sequence descriptions from the system controller 16, notably through an interface circuit 42 included in the system controller 16. As will be appreciated by those skilled in the art, such pulse sequence descriptions generally include digitized data defining pulses for exciting the coils of the gradient coil assembly 24 during excitation and data acquisition phases of operation. Fields generated by the transmit coil assembly 26 excite the spin system within the subject 30 to cause emissions from the anatomy of interest 20. Such emissions may be detected by device 32 or other receive coil (not shown) and are filtered, amplified, and transmitted to signal processing circuit 40. Signal processing circuit 40 may perform preliminary processing of the detected signals, such as amplification of the signals. Following such processing, the amplified signals are transmitted to the interface circuit 42 for further processing.

In addition to the interface circuit 42, the system controller 16 includes central processing circuit 44, memory circuit 46, and interface circuit 48 for communicating with the operator interface station 18. In general, the central processing circuit 44, which will typically include a digital signal processor, a CPU or the like, as well as associated signal processing circuit, command excitation and data acquisition pulse sequences for the magnet assembly 12 and the control and acquisition circuit 14 through the intermediary of the interface circuit 42. The central processing circuit 44 also processes image data received via the interface circuit 42, to perform 2D Fourier transforms to convert the acquired data from the time domain to the frequency domain, and to reconstruct the data into a meaningful image. The memory circuit 46 serves to save such data, as well as pulse sequence descriptions, configuration parameters, and so forth. The interface circuit 48 permits the system controller 16 to receive and transmit configuration parameters, image protocol and command instructions, and so forth.

The operator interface station 18 includes one or more input devices 50, along with one or more display or output devices 52. In a typical application, the input device 50 will include a conventional operator keyboard, or other operator input devices for selecting image types, image slice orientations, configuration parameters, and so forth. The display/output device 52 will typically include a computer monitor for displaying the operator selections, as well as for viewing scanned and reconstructed images. Such devices may also include printers or other peripherals for reproducing hard copies of the reconstructed images.

FIG. 2 is a flow-chart 60 illustrating an exemplary method to reduce RF-induced heating, according to aspects of the present technique for use in the system of FIG. 1. The method includes a step 62 for externally coupling an impedance component to a conducting wire and a step 64 for dynamically varying an impedance of the conducting wire leading to step 68 causing disruption of resonant conditions in the conducting wire and avoiding current and/or voltages built-up on the conducting wire. The impedance is varied, for example, at one end such that the impedance is at a first level when the conducting wire is in a conducting state and at a second level when the conducting wire is in a non-conducting state. It will be easily appreciated by one skilled in the art that the impedance value at the first level is substantially more than the impedance value at second level. The first level may be a ‘high’ impedance value and the second level may be a ‘low’ impedance value. A ‘high’ impedance value may be an impedance value whose magnitude is large compared to a quantity called the characteristic impedance of the conducting wire, and a ‘low’ value would be small compared to this characteristic impedance. However, ‘high’ and ‘low’ impedance values may also be comparable to other impedances in the system, such as the input impedances of the amplifiers, or standard 50 Ohms impedance. Though the ‘high’ and ‘low’ impedance values generally relate to magnitude component of the impedance value, it will also be well appreciated by those skilled in the art that either a phase or the magnitude of the impedance value or the combination of the phase and the amplitude of the impdeance value may be varied at one end of the conducting wire to influence the heating. In one example a time varying impedance is used for earthing (grounding) the conducting wire, as shown at step 66. The method also includes a step 76 of applying a control signal for dynamically varying the impedance of the conducting wire. In one example, the control signal may be an external control input, applied externally in a pre-ordained manner. In another example, the control signal may be a feedback-based control input, for example a feedback from the voltages and/or currents and/or voltages generated on the conducting wire. In one implementation of the method a dynamic impedance circuit may be coupled to a device (via a conducting wire). The impedance may be varied according to a pre-determined control input (or alternately a control logic in the circuit). RF pulses may be applied while the impedance varies thus reducing the current and/or voltage built-up on the conducting wire. The circuit may be disconnected when the RF pulses are not being applied.

FIG. 3 illustrates an exemplary embodiment of a conducting wire assembly 80 for use in the method of FIG. 2. The conducting wire 34 is coupled externally at one end to a time varying impedance component Z (t) denoted generally by reference numeral 82. The impedance component 82 is configured to dynamically vary the impedance of the conducting wire to disrupt resonant conditions of the conducting wire and to avoid current and/or voltage built-up on the wire. The impedance is varied dynamically with time as explained in reference to FIG. 2. In a specific example, the impedance component couples the conducting wire to ground potential, depicted generally by reference numeral 84 and thus effectively alters the boundary condition of the conducting wire. Additionally, the dynamic impedance could be placed elsewhere on the conducting wire, it does not necessarily have to be placed at the end. Examples of impedance components include circuits constructed with inductors and capacitors. Their impedances can be made variable by using variable impedance components (e.g. a device whose capacitance changes with a bias voltage). It will be well understood by those skilled in the art that there are a number of such circuits, for example filters of one form or another.

FIG. 4 illustrates another embodiment 90 for use in the method of FIG. 2. The induced current and/or voltage (and the impedance thereof) on a conducting wire may also be determined by the length of the conducting wire. When a cable has a length of a half wavelength or longer, the induced currents and/or voltages on the cable might be high. On the other hand, when the cable length is smaller than quarter wavelength, the induced currents and/or voltages on the cable are generally small. In some interventional applications, the minimum cable length is much longer than 20 cm. Therefore, it may be advantageous to divide the cables in small electrical segments. The present technique involves use of one or more diodes to alter an electrical length of the cable or a conducting wire, while keeping the physical length intact. In one example, as illustrated in FIG. 4, the impedance component coupled externally to the conducting wire 34 includes a diode or a group of diodes 92, for example PIN diodes, adapted for altering an electrical length of the conducting wire by switching on and off to change the effective length of the end of the wire and therefore to dynamically vary the impedance of the conducting wire. In one example, the one or more diodes are reversed biased diodes having extremely high impedance, effectively breaking the long wires into several electrical segments too short to support the formation of standing waves, and thus avoiding the current and/or voltage built-up and the related heating of tissue and the heating of the device. Again the conducting wire 34 may be grounded as shown generally by reference numeral 84 providing similar benefits as mentioned in reference to FIG. 3. DC-blocking capacitors 94 may be provided between the bias injection points of the diodes 92 so that each diode may be turned on independently.

Both the embodiments of FIG. 3 and FIG. 4 may also include an impedance control circuit 36 (shown in FIG. 4) to trigger the impedance component to vary the impedance via a control signal. The control signal as explained in reference to FIG. 2 may be derived from an external control input that may be a system or user input or the control signal may be derived from a feedback-based control input.

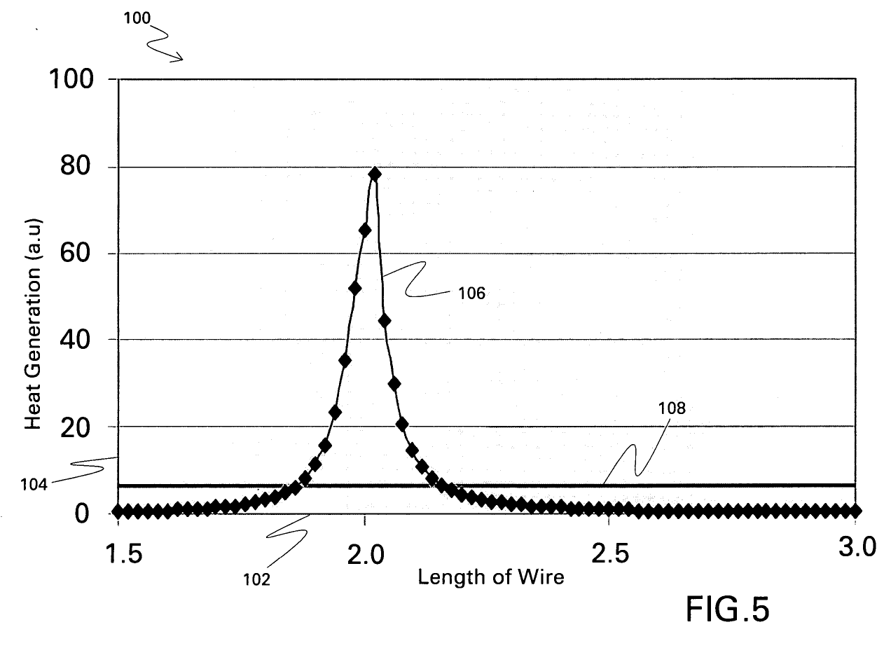

FIG. 5 illustrates through a graph 100, exemplary results for heating effect on the conducting wire by using standard termination of conducting wire compared to dynamically varying impedance as explained herein. The X-axis is denoted generally by reference numeral 102 and denotes the length of wire in meters and the Y-axis is denoted generally by reference numeral 104 and denotes the heating in arbitrary units(a.u.). The curve denoted by reference numeral 106 illustrates a typical thermal profile when any standard termination technique is used for the conducting wire and the line 108 illustrates the thermal profile on the conducting wire using the aspects of present technique. As is clear from the graph 100, aspects of present technique keep the heating within desirable limits irrespective of the length of the wire.

Aspects of the present technique as described herein employ a time-varying change of the electrical properties of one end of the conducting structure to keep the conducting wire out of a stable resonance condition, and thereby reducing the build-up of large currents and/or voltages and the associated heating effects. The advantages offered by the aspects of present technique thus include, providing an RF-safe invasive device with a capability to eliminate or avoid the high currents and/or voltages and excessive electrical fields by preventing the formation of said standing waves. This in turn helps to avoid the risk of tissue heating and device heating due to induced high currents and/or voltages or excessive electrical fields along the conducting wire. Thus it is possible to have a major reduction in heating, with the ability to implement the aspects of this technique as described in FIG. 3 and FIG. 4, on the section of the wire-like device that is not adjacent to or inside the patient's body. In embodiments of the present invention, there is no need for adding structure to the device that is inside the body. In contrast, the current available solutions involve adding structure to the device that is inside the body, which generally interferes with the intended application. For example, such solutions often add diameter to the device that renders it less suitable for negotiating small passageways such as blood vessels.

It will be appreciated by one skilled in the art that the conducting wire may be a conductor placed inside the device used in MR imaging, or it may be incorporated into a coaxial cable which may be a magnetic resonance imaging coaxial cable designed for enhanced safety so as to reduce the risk of excessive heating or burns to a user. The aspects of present technique may be used in other cable types such as single conductor cables, multi conductor cables with and without a shield. The applications are not limited to the cables of the probes that will be inserted into the body. Any cable that has possibility of getting close to the patient will benefit from this design because of the increased safety. These conducting wires, conductors and cables are useful for increasing the safety of RF probes that are inserted into the body such as endorectal, esophageal, intravascular RF probes and interventional surgical devices. As employed herein the term “magnetic resonance imaging” refers to both the use of magnetic resonance apparatus and procedures to generate an image and in spectrographic uses.

The aspects of the present technique may also have applications other than magnetic resonance imaging. The techniques described herein may be used in any circuit that requires low unbalanced currents and/or voltages at a specific operating frequency. For example, if the field pattern of an antenna is affected by the presence of a nearby cable this cable can be provided with dynamically varying impedance described earlier.

While only certain features of the invention have been illustrated and described herein, many modifications and changes will occur to those skilled in the art. It is, therefore, to be understood that the appended claims are intended to cover all such modifications and changes as fall within the true spirit of the invention.

1. A conducting wire assembly for use with a magnetic resonance imaging (MRI) system, the conducting wire assembly comprising:
at least one impedance component coupled externally to a conducting wire, wherein the impedance component is configured to dynamically vary an impedance of the conducting wire to disrupt resonant conditions of the conducting wire and to avoid current and/or voltage built-up on the wire.

2. The conducting wire assembly of claim 1 wherein the impedance component is coupled externally at one end of the conducting wire.

3. The conducting wire assembly of claim 1 wherein the impedance is varied dynamically with time such that the impedance is at a first level when the conducting wire is in a conducting state and at second level when the conducting wire is in a non-conducting state, wherein the impedance component couples the conducting wire to ground potential.

4. The conducting wire assembly of claim 3 wherein an impedance value at the first level is substantially more than the impedance value at second level.

5. The conducting wire assembly of claim 1 wherein the impedance component comprises a diode adapted for altering an electrical length of the conducting wire to dynamically vary the impedance of the conducting wire.

6. The conducting wire assembly of claim 1 further comprising an impedance control circuit to trigger the impedance component to vary the impedance via a control signal.

7. The conducting wire assembly of claim 6 wherein the control signal is derived from an external control input.

8. The conducting wire assembly of claim 6 where the control signal is derived from a feedback-based control input.

9. An invasive device comprising:
at least one conductor assembly, the conductor assembly comprising an impedance component coupled externally to a conductor, wherein the impedance component is configured to dynamically vary an impedance of the conductor to disrupt resonant conditions of the conductor to avoid current and/or voltage built-up on the conductor; and
an impedance control circuit to trigger the impedance component via a control signal to vary the impedance.

10. The invasive device of claim 9 wherein the impedance component is coupled externally at one end of the conductor.

11. The invasive device of claim 9 wherein the impedance component comprises a diode adapted for altering an electrical length of the conductor to dynamically vary the impedance of the conductor.

12. The invasive device of claim 9 further comprising an impedance control circuit to trigger the impedance component to vary the impedance via a control signal.

13. The invasive device of claim 12 wherein the control signal is derived from an external control input.

14. The invasive device of claim 12 where the control signal is derived from a feedback-based control input.

15. A magnetic resonance imaging (MRI) cable comprising:
at least one conducting wire assembly, the conducting wire assembly comprising an impedance component coupled externally to a conducting wire, wherein the impedance component is configured to dynamically vary an impedance of the conducting wire to disrupt resonant conditions of the conducting wire and to avoid current and/or voltage built-up on the conducting wire.

16. The MRI cable of claim 15 wherein the impedance component comprises a diode adapted for altering an electrical length of the conducting wire to dynamically vary the impedance of the conducting wire.

17. The MRI cable of claim 15 further comprising an impedance control circuit to trigger the impedance component to vary the impedance via a control signal.

18. The MRI cable of claim 17 wherein the control signal is derived from an external control input.

19. The conducting wire assembly of claim 17 where the control signal is derived from a feedback-based control input.

20. A magnetic resonance imaging (MRI) system comprising:
an array of radio frequency coils for producing controlled gradient field and for applying excitation signals to a volume of interest;
a device incorporating a conducting wire for detecting magnetic resonance signals resulting from the excitation signals applied to the volume of interest;
an impedance component coupled externally to the conducting wire, wherein the impedance component is configured to dynamically vary an impedance of the conducting wire to disrupt resonant conditions of the conducting wire and to avoid current and/or voltage built-up on the conducting wire;
a control and acquisition circuit configured to energize the array of radio frequency coils and for triggering the impedance component via a control signal to dynamically vary the impedance; and
a system controller circuit configured to acquire an image from the magnetic resonance signals detected by the device.

21. The MRI system of claim 20 wherein the impedance component is coupled externally at one end of the conducting wire.

22. The MRI system of claim 20 wherein the impedance component comprises a diode adapted for altering an electrical length of the conducting wire to dynamically vary the impedance of the conducting wire.

23. The MRI system of claim 20 further comprising an impedance control circuit to trigger the impedance component to vary the impedance via a control signal.

24. The MRI system of claim 23 wherein the control signal is derived from an external control input.

25. The MRI system of claim 23 where the control signal is derived from a feedback-based control input.

26. A method for reducing radiofrequency (RF)-induced heating in magnetic resonance imaging (MRI), the method comprising:
externally coupling an impedance component to a conducting wire;
dynamically varying an impedance of the conducting wire with time via the impedance component for disrupting resonant conditions in the conducting wire and for avoiding current and/or voltage built-up on the conducting wire.

27. The method of claim 26 further comprising varying the impedance at one end of the conducting wire, wherein the impedance is at a first level when the conducting wire is in a conducting state and at a second level when the conducting wire is in a non-conducting state.

28. The method of claim 27 wherein an impedance value at the first level is substantially more than the impedance value at second level.

29. The method of claim 26 further comprising using a time varying impedance for grounding the conducting wire.

30. The method of claim 26 further comprising applying a control signal for dynamically varying the impedance of the conducting wire.

31. The method of claim 30 wherein the control signal is an external control input.

32. The method of claim 30 wherein the control signal is a feedback-based control input.
